(19) United States
(12) Patent Application Publication
(10) Pub. No.: US 2024/0185992 A1
(43) Pub. Date: Jun. 6, 2024

(54) Device For Supporting A Patient With A Chronic Or A Non-Chronic Disease And Method For Operating The Device

(71) Applicant: Roche Diabetes Care, Inc., Indianapolis, IN (US)

(72) Inventors: Matthias Essenpreis, Basel (CH); Dirk Voelkel, Weinheim (DE); Stefan Weinert, Pendleton, IN (US)

(73) Assignee: Roche Diabetes Care, Inc., Indianapolis, IN (US)

(21) Appl. No.: 18/414,601

(22) Filed: Jan. 17, 2024

Related U.S. Application Data
(63) Continuation of application No. 13/586,409, filed on Aug. 15, 2012, now abandoned.

(30) Foreign Application Priority Data
Aug. 16, 2011 (EP) 111777124

(51) Int. Cl. G16H 40/20; G16H 40/63

(52) U.S. Cl. CPC G16H 40/20; G16H 40/63

(57) ABSTRACT

A device and method are disclosed for supporting a patient with a disease, comprising a display facility, a user interface, a processor, and a user interface, and with an application module comprising program instructions, which are configured, on executing by the processor to bring about the following: providing medical protocols for diagnostic and/or therapeutic purposes, collecting time information concerning a future event which is a trigger for at least one medical protocol, selecting one of the medical protocols which indicates a group of cohesive protocol events which are spaced apart chronologically over a protocol period in accordance with a protocol regime, monitoring a chronological interval between a current date and the future even, and issuing a user start notice concerning the beginning of the medical protocol at a time which from a chronological point of view lies at least by the protocol period before the future event.

### REFERENCE

This application is based on and claims priority to European Patent Application No. EP 11177712.4 filed Aug. 16, 2011, which is hereby incorporated by reference.

### FIELD

The present disclosure relates to a device for supporting a patient with a chronic or a non-chronic disease, in particular diabetes, and a method for operating the device.

### BACKGROUND

Such devices are provided in order to facilitate the patient's dealing with and coping with the disease. However, such devices also serve the treating doctor in attending to diagnostic or therapeutic measures in a supportive manner. The devices do not intervene here into the diagnostics or therapy itself or participate directly in this, but rather serve in particular to collect and process electronic information, in particular in the form of measurement data, and to produce data outputs deriving therefrom.

From the document WO 2010/072386 A2 a structured measurement method for diagnostic or therapeutic purposes for a patient with a chronic disease and a device for using the method are known. It is proposed to select and implement a structured collection protocol for the diagnostic or therapeutic support of a patient automatically by means of an application implemented on a device, wherein the structured collection protocol defines the collection procedure by means of associated parameters. For example, a schedule for events for measurement data acquisition belongs to the parameters.

The document US 2008/0201174 A1 discloses a personalized medical management system by which automatically reminders can be set and generated for a user, which are triggered by an event. Thus, in the light of a pending consultation with a doctor, an automatic reminder can be issued to the user, which indicates specific requirements in connection with the future appointment. Thus, the user can be reminded that he is to arrive for the consultation having fasted.

The document US 2003/0211617 A1 discloses a method and a device for implementing a blood glucose meter, which reminds the user of the necessary measuring of a blood glucose value. This takes place in relation to a previously acquired measured value.

The document WO 2010/072386 A2 discloses an apparatus for supporting the diagnosis or therapy of a chronic disease of a patient. The apparatus has a display facility, a user interface and a processor which is coupled to the display facility and the user interface. Program instructions are provided, which are carried out by the processor and comprise for example the providing of medical protocols for diagnostic and/or therapeutic purposes. Time information concerning a future event can be detected as a trigger for a medical protocol. A selection of one of the medical protocols is provided here, which indicates a group of cohesive protocol events which are spaced apart chronologically over a protocol period. The apparatus comprises in addition the issuing of a user start notice concerning the beginning of a medical protocol.

In document EP 2 348 470 A1, a device and a method are disclosed for providing reminders for a user.

The document US 2002/0029161 A1 describes a method, a computer program and a system for assigning resources to tasks in a rule based system.

### SUMMARY

Improved technologies for supporting a patient with a chronic or a non-chronic disease, in particular diabetes, are disclosed by which diagnostic or therapeutic measures can be attended supportively in an improved manner.

A device for supporting a patient with a chronic or a non-chronic disease according to the independent claim 1, and a method for operating the device according to the independent claim 15 are provided. Further embodiments are the subject matter of dependent claims.

A device for supporting a patient with a chronic or a non-chronic disease, in particular diabetes, is provided, comprising a display facility, a user interface, a processor which couples to the display facility and the user interface with respect to data technology, and comprising an application module comprising program instructions which are configured on executing by the processor to bring about the following:
providing medical protocols for diagnostic and/or therapeutic purposes;
collecting time information concerning a future event, which is a trigger for at least one medical protocol;
selecting one of the medical protocols, which indicates a group of cohesive protocol events, which are spaced apart chronologically over a protocol period in accordance with a protocol regime;
monitoring a chronological interval between a current date and the future event; and
issuing a user start notice concerning the beginning of the medical protocol at a time which from a chronological point of view lies at least by the protocol period before the future event.

Further, a method is provided for operating a device for supporting a patient with a chronic or a non-chronic disease, in particular diabetes, comprising a display facility, a user interface, a processor which couples to the display facility and the user interface with respect to data technology, and comprising an application module comprising program instructions, which are configured, wherein the method comprises the following steps on executing the program instructions by the processor:
providing medical protocols for diagnostic and/or therapeutic purposes;
collecting time information concerning a future event, which is a trigger for at least one medical protocol;
selecting one of the medical protocols, which indicates a group of cohesive protocol events, which are spaced apart chronologically over a protocol period in accordance with a protocol regime;
monitoring a chronological interval between a current date and the future event; and
issuing a user start notice concerning the beginning of the medical protocol at a time which from a chronological point of view lies at least by the protocol period before the future event.

As a function of a future event, for example, a medical consultation appointment such as a doctor's appointment, for which time data are stored in electronic form which characterize the future event from an appointment-related or chronological point of view, and as a function of a selected medical protocol, the user of the device, i.e. a patient with a chronic or non-chronic disease, is automatically notified of the start of a carrying out or executing of a protocol necessary before the future event, i.e. for example the doctor's appointment, in accordance with the protocol regime. The medical protocol is the trigger or reason for carrying out the medical protocol.

The medical protocol can be, for example, a structured measured value acquisition protocol, which indicates a group of cohesive measurement events which are spaced apart chronologically over a measurement period in accordance with a measurement regime. However, the alternative or supplementary logging of other events can be the subject matter of the medical protocol, of which for example the logging of sports activities and/or food intake by the patient can be a part.

The date for the future event on the one hand and the medical protocol on the other hand determine the time for the generating and issuing of the user start notice, by which the user is informed of the beginning of the medical protocol. It is essential here that the issuing of the user start notice takes place punctually at a time which, from a chronological point of view lies at least by the protocol period before the date for the future event, so that the time for the issuing of the user start notice depends on the selected medical protocol.

In this way, for example, in the case of a measured value acquisition protocol, it is ensured that the user measures and acquires the necessary measurement data punctually and in the desired structured form for the future event, which is, for example, a medical consultation or doctor's appointment and which is the trigger for the acquisition protocol. The patient can collect the measurement data itself in any desired manner, for example by noting this into a paper protocol in accordance with the structured measured value acquisition protocol. However, the storage of the measured values in electronic form can also be provided in any desired storage medium.

The automatic issuing of the user start notice supports for example diagnostic or therapeutic measures of the doctor or of other medical personnel, by the patient, for example before a pending consultation with the doctor, being instructed and guided to carry out a desired protocol, i.e. to collect the desired measured values precisely in the manner as specified by the structured measured value acquisition protocol.

In so far as the medical protocol is a measured value acquisition, or measured value protocol, the acquired measured values can be, in particular, blood glucose measured values of a diabetes patient. In particular, for such patients it is of great importance to collect the measured values in a structured manner, i.e. in accordance with a specified regime, and in a coordinated manner from a chronological point of view. Only in this way, for example, can the treating doctor derive conclusions from the collected measured data, in order to suggest, where applicable, modified or new therapeutic measures.

The issuing of the user start notice takes place, for example, via the display facility of the device. Thus, a visual notification can be issued with corresponding text via the display facility. In addition or alternatively, the issuing of the user start notice can be provided by means of an acoustic and/or a tactile signaling. The latter can comprise, for example, a vibration alarm.

Provision can, however, also be made to transfer the user start notice via an interface to another output device, whether via a cable-based or a wireless data connection. In this way, it is made possible to implement the application module, for example, in a server facility, which then transfers the user start notice via a corresponding data interface to a user facility, for example a laptop, a mobile radio communication device or a blood glucose meter. In this respect, the processor and display facility can also be implemented in separate pieces of equipment which are coupled with respect to data technology, for which reason in this and other embodiments also the designation system for supporting a patient with a chronic or a non-chronic disease, in particular diabetes, is applicable.

The selection of the medical protocol, for example of the structured measured value acquisition protocol, takes place in a user-specific manner, wherein in this connection provision can be made to evaluate additional information acquired via the user interface, which is specified for example by the treating doctor. However, provision may also be made to acquire user data from the patient, for example age and/or weight, within the application module before the selection of the medical protocol, in order to be able to select, proceeding therefrom, an allocated structured protocol in a patient-specific manner.

In an embodiment, provision can be made to give the user the possibility of adapting or altering a selected medical protocol after the selection from a group of protocols, for example a number of medical standard protocols. The user can thus, for example, add additional protocol events himself. In this way, a more extensive user-specific development of the protocol regime is made possible.

A number of medical protocols can be stored electronically in a memory, which is implemented by the processor in the same unit of equipment. However, a remote access of the processor to a server facility with several medical protocols via a wireless or a cable-related data communication connection can be provided in an embodiment.

The time for issuing the user start notice can already be specified in the medical protocol itself.

Provision can be made to represent visually on a display the information acquired according to the medical protocol, in particular measured values, whether for example on a display of the device itself and/or after a data transmission to a data processing facility of any desired type. Evaluation algorithms can also be used in connection with the acquired protocol data, for example for the evaluation of acquired measured values. This can take place on the device itself and/or on another data processing facility, for example on a central server.

According to a further embodiment, the program instructions are further configured to bring about the issuing of the user start notice on executing by the processor, when on monitoring of the chronological interval between the current date and the future event it is established that a chronological minimum interval between the current date and the future event is reached or prior to being reached. In addition, provision can be made that an advance notice is issued at a particular time before the chronological minimum interval. Hereby, the patient can be notified for example in advance of a subsequently upcoming measured value acquisition.

In an embodiment, the chronological minimum interval can be indicated in the selected medical protocol. In this embodiment, the various medical protocols all or partly already indicate a chronological minimum interval at which the issuing of the user start notice is to take place specifically for a particular protocol.

According to a further embodiment, the program instructions are further configured, on executing by the processor, to bring about the determining of the chronological minimum interval proceeding from the protocol period indicated in the selected medical protocol. In this embodiment, provision is provided in a configuration that on carrying out the instructions of the application module the electronic information is evaluated over the protocol period in the medical protocol. Proceeding therefrom, the chronological minimum interval is determined, which must be at least equal to the protocol period. For the issuing of the user start notice, however, a chronological advance can also be determined by a period being added to the protocol period, for example hours or days or a weekend. The protocol period can, for example, comprise several days, three to five days for example, for which reason the protocol entries are to be carried out advantageously within three to five days before the future event triggering the protocol, for example a consultation with a doctor.

For example, the program instructions can further be configured to bring about, on executing by the processor, the determining of the chronological minimum interval, taking into consideration user-specific information. Thus, provision can be made, in the determining of the chronological minimum interval, which is determined taking into consideration the protocol period indicated in the medical protocol, to incorporate user-specific data which were optionally required previously from the user or are already present in electronic form. Thus, provision can be made, in the determining of the chronological minimum interval, to add a period of time to the protocol period, which depends on the age and/or indicators for the disease status of the patient. Provision can also be made to take specific life circumstances of the user into consideration, for example employment in shift work.

In still a further embodiment, the program instructions are further configured, on executing by the processor after the time for the user start notice and before a future event to bring about the issuing at least of one reminder start notice concerning the beginning of the protocol period. Times for the issuing of the at least one reminder start notice can already be indicated in the medical protocol. Otherwise, the explanations given above regarding variant configurations, in connection with the chronological timing of the issuing of the user start notice, apply accordingly for the issuing of the at least one reminder start notice.

According to a further embodiment, the user start notice and/or the at least one reminder start notice comprise information relating to a start time for the protocol regime according to the selected medical protocol. In this configuration, provision is made for example that the user start notice and/or the at least one reminder start notice indicate the start time with day and time.

For example, the program instructions can further be configured, on executing by the processor for the protocol events to bring about respectively the issuing of a user event notice, which is allocated to one of the protocol events, concerning the pending nature of a protocol event. For the case where the protocol event is a measured value acquisition, the user event notice, which is then a user measurement notice, comprises for example a notice concerning measurement conditions. Thus, the user can be notified for example not to take in any food for a particular time before the measurement. The time or the chronological interval for the issuing of the user event notice, which is allocated to one of the events to be executed according to the protocol, for example measured value acquisitions, can already be appointed in the selected medical protocol. Otherwise, the comments made previously in connection with configurations concerning the user start notice apply accordingly.

In an embodiment, the user event notice in accordance with allocated event information in the medical protocol refers to a user-specific event existing chronologically before or after the protocol event. User-specific events can be, for example, a meal and/or a sports activity of the patient. The user event notice then gives the patient protocol-specific notices as to how the protocol event concerned, i.e., for example a measured value acquisition, stands in chronological relation to the user-specific event. Thus, for example, a fixed chronological interval can be indicated, by which a measurement, which is to be recorded, is to be carried out before and/or after the user-specific event, for example a chronological interval from an intake of food.

In still another embodiment, the program instructions are further configured, on executing by the processor after the time of issuing of the user event notice and before an event time for the protocol events allocated to the protocol event respectively to bring about the issuing at least of one reminder event notice which is allocated to the protocol event, concerning the pending nature of the protocol event. This means, for example, that the user is firstly notified of a protocol event (user event notice), for example a pending measurement. Before the measurement is then actually pending, the user is reminded of this at least once (reminder event notice). Time information defining the issuing of the at least one reminder event notice can be already comprised by the medical protocol. Otherwise, the explanations given in connection with the user start notice and/or with the at least one reminder start notice with regard to different embodiments also apply here accordingly.

According to an embodiment, the user event notice and/or the at least one reminder event notice comprise information relating to the event time. The explanations given above in connection with the user start notice and/or with the at least one reminder start notice apply here accordingly.

In a further embodiment, the program instructions are further configured, on executing by the processor, to bring about collecting and electronically storing protocol information associated with the protocol events. In this embodiment, provision can be made to make available to the patient via the user interface and/or the display facility a protocol acquisition mask, for example a measured value acquisition mask, in particular for the structured acquisition of blood glucose measured values. Provision can be made optionally here to acquire supplementary user-specific information, for example concerning meals and/or sports activities, for which a chronological relationship to the measured values can exist.

According to an embodiment, the medical protocols are formed as structured measured value acquisition protocols which respectively indicate a group of cohesive measurement events which are spaced apart chronologically over a measurement period according to a measurement regime, and wherein the user start notice relates to the beginning of a measured value acquisition corresponding to the structured measured value acquisition protocol at a time which from a chronological point of view lies at least by the measurement period before the future event.

In still another embodiment, the processor is formed in an apparatus selected from the following apparatuses: data processing facility such as a laptop, data communication device such as mobile telephone and medical measurement- or analysis apparatus such as blood glucose meter. The device or the system for supporting a patient with a chronic/non-chronic disease can take on various characteristics. The application module with the program instructions can be implemented by means of suitable software in different apparatus types or -systems. This also includes, for example, applications on mobile telephones. However, medical- or analysis apparatuses themselves can also realize the described management of the protocol data acquisition with respect to a future doctor's appointment.

In connection with the method for operating a device for supporting a patient with a chronic or a non-chronic disease, in particular diabetes, the explanations given above in connection with the embodiments of the device for supporting the patient apply accordingly.

### BRIEF DESCRIPTION OF THE DRAWINGS

Exemplary embodiments are described in the following with reference to figures of drawings.

FIG. 1 shows a diagrammatic illustration of an arrangement with a device for supporting a patient of a chronic or of a non-chronic disease, in particular diabetes;

FIG. 2 shows a diagrammatic block illustration concerning process steps on processing of program instructions in a processor; and

FIG. 3 shows a diagrammatic timeline illustration concerning process steps on processing of program instructions in a processor.

### DETAILED DESCRIPTION

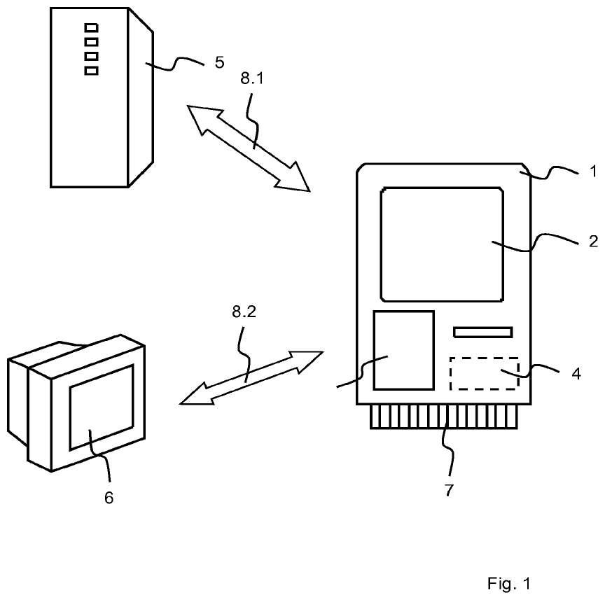

FIG. 1 shows a diagrammatic illustration of an arrangement with a device 1 for supporting a patient of a chronic or a non-chronic disease, in particular diabetes. The device 1 has a display facility 2 in the form of a display, a user interface, which in the illustrated embodiment has a keyboard 3, and a processor 4, which is connected with the display facility 2 and with the keyboard 3 with respect to data technology, so that electronic data are able to be exchanged between the elements. In the arrangement according to FIG. 1 a server facility 5 and a further display facility 6 are additionally shown, which couple functionally to the device 1 such that electronic data are able to be exchanged via an associated interface 7 of the device 1 and a wireless and/or cable-based communication connection 8.1, 8.2.

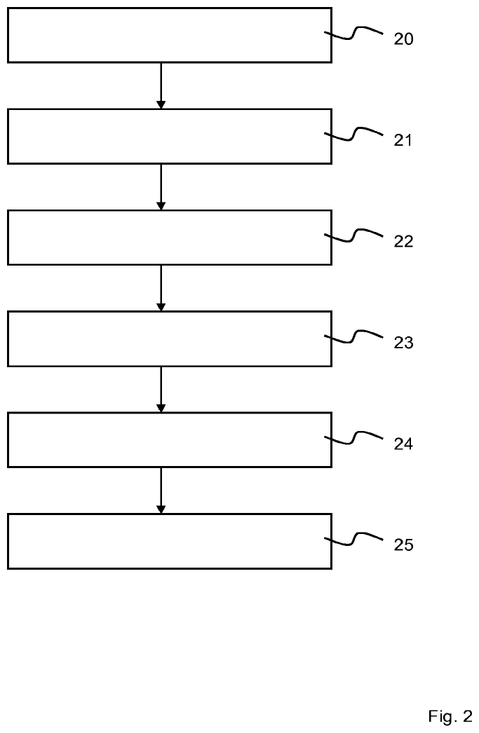

The device 1 is configured by means of hard- and software to support a patient with a chronic or a non-chronic disease in the following manner. For this, reference is made for an embodiment to a diagrammatic illustration in FIG. 2.

The device 1 can implement different types of apparatus, for example a mobile computer, a mobile telephone or a blood glucose meter. With the aid of a software-related application module which comprises program instructions, the device 1 configured, on executing the program instructions with the processor 4 to bring about the following. In a step 20, time information is collected concerning a future medical concentration of a patient, i.e. for example time information for a future doctor's appointment. The acquisition of the time information can take place for example in that the user or patient inputs the time data by means of the keyboard 3.

In the following step 21, by means of the processor 4 a structured measured value protocol is selected. This selection can be carried out from a number of structured measured value acquisition protocols which are stored in the device 1 itself. However, an access to a number of structured measured value acquisition protocols in the server facility 5 can take place here. The structured measured value acquisition protocol indicates in particular a number of cohesive measurement events, which are spaced apart chronologically over a measurement period according to a protocol-specific regime. The measurement period can extend over one or several days. Basically, any desired measurement periods are possible. The structured measured value acquisition protocol indicates how many measurement events are to take place in the indicated measurement period. Furthermore, the time for the measurement events is specified by the structured measured value acquisition protocol. This can take place by concrete date and time information. Provision may also be made to set the measurement events, i.e. the acquisition of measured values by the patient, in relation to the user-specific events, for example a meal or a sports activity of the user.

Then in a step 22 of the device 1 the chronological interval between a continuing current date and the date for the future consultation with the doctor is monitored.

If it is established here that the chronological interval between the current date and the future doctor's appointment is reached or prior to being reached, then in a step 23 a user start notice is generated and issued by the processor. The user start notice gives the patient information concerning the time of the start of the measured value acquisition in accordance with the structured measured value acquisition protocol. This means that the user is informed as to when the measured value process, associated with the future doctor's appointment, has to begin in accordance with the structured measured value acquisition protocol. The issuing of the user start notice can take place via the display facility 2 and/or the further display facility 6. This means that the issuing of the user start notice can take place through the device 1 itself and/or through an apparatus which is separate therefrom.

In a step 24 provision is optionally made to give the user of the device 1 one or more reminder start notices between the time of the issuing of the user start notice and the actual start time of the measured value acquisition, in order to remind the patient of the pending measured value acquisition in accordance with the structured measured value acquisition protocol.

In a step 25 according to FIG. 2, finally, a user measurement notice is generated and issued, by which the user of the device 1 is now notified of an actual measuring in accordance with the structured measured value acquisition protocol.

The collecting of the measured value data can then take place in various ways. Thus, provision can be made to make a measurement protocol available to the patient in paper form, into which he notes in writing the measured values which are measured in accordance with the structured measured value acquisition protocol, for example in the form of blood glucose measured values. Alternatively or additionally, provision can be made that by means of the device 1 and/or a data processing system separate therefrom, an electronic measurement protocol mask is provided, into which the user then enters the measured values. Irrespective of the actual nature of collecting and storing of the measured values, information is then available for the doctor's appointment, so that the medical personnel can evaluate this information. It is thus ensured that the measured value acquisition takes place in the desired manner, in particular with regard to the frequency and the chronological interval between the measurement events.

Exemplary embodiments are disclosed in connection in particular with a structured measured value acquisition protocol. The aspects of the disclosure can, however, also be used with different medical protocols, which serve diagnostic and/or therapeutic purposes. Thus, alternatively or additionally, other protocol events can be acquired in accordance with a medical protocol, for example an intake of medicament, an intake of food and/or symptom observations of the patient.

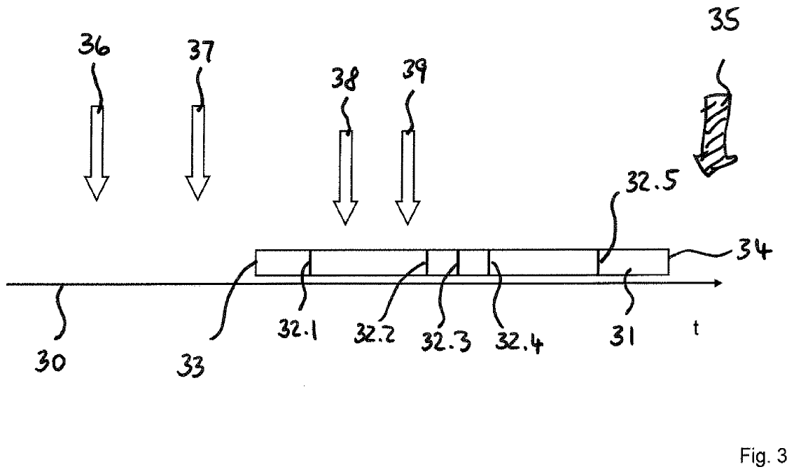

FIG. 3 shows a diagrammatic illustration of the chronological sequence of process steps. A protocol period 31 and protocol events 32.1, . . . , 32.5 lying herein are represented along a timeline 30. The protocol period 31 extends from a start of the protocol 33 up to the end of the protocol 34. The trigger for the medical protocol, i.e. for example a measured value acquisition protocol, in which the protocol events 32.1, . . . , 32.5 correspond to a respective measured value acquisition, is a future event 35, which is for example a medical consultation appointment with a doctor. Before the start of the protocol 33, a user start notice 36 is issued. The user is informed in this way of the pending medical protocol. In accordance with the diagrammatic representation in FIG. 3, a reminder to the user of the pending medical protocol is provided by a reminder start notice 37.

During the protocol period, provision is then also made in the configuration in FIG. 3 to inform the user of a particular protocol event 32.2 with a user event notice 38. For example, the user is informed in this way of a pending blood glucose measurement. This can take place for example in visual and/or acoustic form of signaling. In the illustrated example embodiment in FIG. 3, in addition a reminder event notice 39 is then provided, by which the user is reminded of the pending protocol event 32.2.

As can be seen from FIG. 3, the protocol events 32.1, . . . , 32.5 can have different or identical chronological intervals from one another.

Thus, embodiments of the device for supporting a patient with a chronic or a non-chronic disease and method for operating the device are disclosed. One skilled in the art will appreciate that the teachings can be practiced with embodiments other than those disclosed. The disclosed embodiments are presented for purposes of illustration and not limitation, and the invention is only limited by the claims that follow.

1.-15. (canceled)

16. A method for operating a device for supporting a patient with diabetes, including a display facility, a user interface, a processor which couples to the display facility and the user interface with respect to data technology, and including an application module having non-transient program instructions stored in a computer memory on the device and configured to cause the processor to perform the following steps:
providing a plurality of medical protocols for diagnostic or therapeutic purposes in the computer memory, wherein each of the plurality of medical protocols is a structured measured value acquisition protocol, which indicates a group of cohesive measurement events which are spaced apart chronologically over a measurement period in accordance with a measurement regime, wherein the measurement period extends from a beginning of the medical protocol to an end of the medical protocol, and wherein each of the plurality of medical protocols indicates a protocol period at least equal to the measurement period,
collecting time information concerning a date of a future event, wherein the future event is a trigger for at least one medical protocol from the plurality of medical protocols,
receiving input from the patient via the user interface, where the input includes user-specific information from the patient and the user-specific information is additional to any information regarding the future event,
selecting one of the medical protocols from the plurality of medical protocols, which indicates the protocol period before the date of the future event to begin executing the selected medical protocol, wherein each structured measured value acquisition protocol indicates how many measurements events are to take place in the period and a time at which each measurement event is to occur, wherein the measured values for the measurement events are blood glucose measurements,
determining a chronological minimum interval by adding a period of time to the protocol period, where the period of time depends on the user specific information,
monitoring a chronological interval between a current date and the future event, and
issuing, via the display facility, a user start notice for the beginning of the selected medical protocol before the date of the future event, where the user start notice is issued at the chronological minimum interval before the date of the future event.
